(19) United States
(12) Patent Application Publication
(10) Pub. No.: US 2024/0108528 A1
(43) Pub. Date: Apr. 4, 2024

(54) EXERCISE REHABILITATION HARNESS FOR ORTHOTIC BOOT

(71) Applicant: Benjamin SCHUMACHER, Champlin, MN (US)

(72) Inventor: Benjamin SCHUMACHER, Champlin, MN (US)

(21) Appl. No.: 18/374,134

(22) Filed: Sep. 28, 2023

Related U.S. Application Data
(60) Provisional application No. 63/413,080, filed on Oct. 4, 2022.

(51) Int. Cl. A61H 1/00; A61F 5/01

(52) U.S. Cl. CPC A61H 1/00; A61F 5/0195

(57) ABSTRACT

An exercise rehabilitation harness to secure about an orthotic boot includes a toe cap to receive a toe portion of the boot. A heel strap includes a heel strap coupling member to be disposed on a backside of a heel portion of the boot, and first and second heel strap segments to extend along opposing sides of a foot portion of the boot from the toe cap to second ends to be interconnected by the heel strap coupling member. An ankle strap includes an ankle strap coupling member and an ankle strap segment to wrap around an ankle portion of the boot vertically above the heel strap with opposing ends to be interconnected by the ankle strap coupling member. A connector element to connect to exercise equipment is attached to the ankle strap segment so as to be disposed on a backside of the ankle portion of the orthotic boot.

### CROSS REFERENCE TO RELATED APPLICATION

This Non-Provisional patent application claims the benefit of the filing date of U.S. Provisional Patent Application Ser. No. 63/413,080, filed Oct. 4, 2022, which is herein incorporated by reference.

### TECHNICAL FIELD

The present disclosure relates generally to exercise equipment and, in particular, to an exercise rehabilitation harness for use with an orthotic boot.

### BACKGROUND

Orthotic boots are common for treating fractures and other injuries of the lower leg and foot. Orthotic boots, which go by many names, such as walking boots, walking casts, stabilizers, and air casts, to name a few, are specifically designed to immobilize specific regions along the ankle and leg. By immobilizing the calf, ankle, and foot, orthotic boots keep the injured parts in place and prevent them from moving to promote healing.

### SUMMARY

Examples of the present disclosure provide a rehabilitation harness for use with an orthotic boot.

One example provides an exercise rehabilitation harness to secure about an orthotic boot. The exercise rehabilitation harness includes a toe cap defining a toe cup to receive a toe portion of the orthotic boot, and a heel strap including a heel strap coupling member to be disposed on a backside of a heel portion of the orthotic boot, a first heel strap segment and a second heel strap segment, the first and second segments each having a first end connected to the toe cap, the first and second heel strap segments extending longitudinally in parallel with one another on opposite sides of a foot portion of the orthotic boot to second ends, the heel strap coupling member to interconnect the second ends on the backside of the heel portion of the orthotic boot. An ankle strap includes an ankle strap coupling member, and an ankle strap segment having opposing first and second ends, the ankle strap segment to wrap around and secure to an ankle portion of the orthotic boot vertically above the heel of the orthotic boot, the ankle strap coupling member to interconnect the opposing first and second ends. A connector element is attached to the ankle strap segment so as to be disposed on a backside of an ankle portion of the orthotic boot, the connect element to connect to exercise equipment. At least one vertically extending connector strap connects the ankle strap segment to one of the first and second heel strap segments.

One example provides an exercise rehabilitation harness to secure about an orthotic boot, the harness including a toe cap defining a toe cup to receive and fit over a toe portion of the orthotic boot, the toe cap having a toe connector element. The toe connector element is disposed at a location such that when the exercise harness is secured to the orthotic boot and a lower leg and foot of a user are secured within the boot, the toe connector element is to be positioned along a longitudinal access of the foot and be centered and forward of phalanges of the foot, the toe connector element to connect to exercise equipment. A heel strap includes a heel strap coupling member to be disposed on a backside of a heel portion of the orthotic boot; a first heel strap segment and a second heel strap segment, the first and second segments each having a first end connected to the toe cap, the first and second heel strap segments to extend longitudinally in parallel with one another on opposite sides of a foot portion of the orthotic boot to second ends. The heel strap coupling member to interconnect the second ends on the backside of the heel portion of the orthotic boot.

Additional and/or alternative features and aspects of examples of the present technology will become apparent from the following description and the accompanying drawings.

### BRIEF DESCRIPTION OF THE DRAWINGS

FIG. 1 is a right front perspective view generally illustrating an exercise rehabilitation harness for use with an orthotic boot, according to one example.

FIG. 2 is a left front perspective view generally illustrating an exercise rehabilitation harness for use with an orthotic boot, according to one example.

FIG. 3 is a right side view generally illustrating an exercise rehabilitation harness for use with an orthotic boot, according to one example.

FIG. 4 is a left side view generally illustrating an exercise rehabilitation harness for use with an orthotic boot, according to one example.

FIG. 5 is a front side view generally illustrating an exercise rehabilitation harness for use with an orthotic boot, according to one example.

FIG. 6 is a rear side view generally illustrating an exercise rehabilitation harness for use with an orthotic boot, according to one example.

FIG. 7 is a bottom side view generally illustrating an exercise rehabilitation harness for use with an orthotic boot, according to one example.

FIG. 8A generally illustrates a top view of the skeletal structure of a human foot.

FIGS. 8B and 8C respectively illustrate lateral and anterior views of the skeletal structure of a human lower leg and foot.

FIG. 9 is a side view generally illustrating an exercise rehabilitation harness secured to an orthotic boot and connected to exercise equipment, according to one example.

Throughout the drawings, identical reference numbers designate similar, but not necessarily identical, elements. The figures are not necessarily to scale, and the size of some parts may be exaggerated to more clearly illustrate the example shown. Moreover the drawings provide examples and/or implementations consistent with the description; however, the description is not limited to the examples and/or implementations provided in the drawings

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense. It is to be understood that features of the various examples described herein may be combined, in part or whole, with each other, unless specifically noted otherwise.

Lower leg injuries (e.g., to the calf, ankle, and foot), such as fractures and tendon sprains/tears, for example, are frequently treated using orthotic boots. While there are different types of orthotic boots, in general, orthotic boots function to immobilize one or more of the calf, ankle, and foot to stabilize an injury in order to promote improved and quicker healing. While aiding with injury recovery, the loss of mobility associated with wearing an orthotic boot for extended periods to time may result in muscle atrophy, even in parts of body not immobilized the orthotic boot, such as in the upper leg and hip region, for example. Upon discontinuing use of the orthotic boot, such muscle atrophy, if it occurs, can potentially increase the risk of re-injury and/or additional injury, and can extend recovery time as the atrophied muscles are strengthened.

In accordance with the present application, as will be described in greater detail herein, a rehabilitation harness is disclosed which, according to examples, includes a toe cap and a number of straps which are adapted to fit over and adjustably secure to an orthopedic boot. The rehabilitation harness includes a number of coupling points for connecting to any number of different types of exercise and/or rehabilitation equipment, including to strength training equipment, such as to cables of adjustable weight stack machines, elastic athletic bands, and Keiser™ machines (to name a few), for example, to enable a patient to perform any number of various exercises, including strength training exercises, when wearing an orthotic boot. Such strength training reduces and/or eliminates muscle atrophy to provide quicker and improved recovery from injury.

While known foot harnesses are available for use with exercise equipment, such known harnesses are typically designed for use with athletic shoes, and are not intended for use (or even capable for use) with orthopedic boots nor intended for injury rehabilitation in general as placement of straps and coupling points may cause forces to be applied to the lower leg and foot that could potentially aggravate injuries. In contrast, the rehabilitation harness of the present application, as will be described in greater detail herein, is configured to securely attach about an orthopedic boot with the straps and connection points configured to be strategically positioned relative to the skeletal and muscular structure of the lower leg, foot, and ankle so as to reduce and/or eliminate torsional and other forces that can otherwise aggravate or exacerbate injuries.

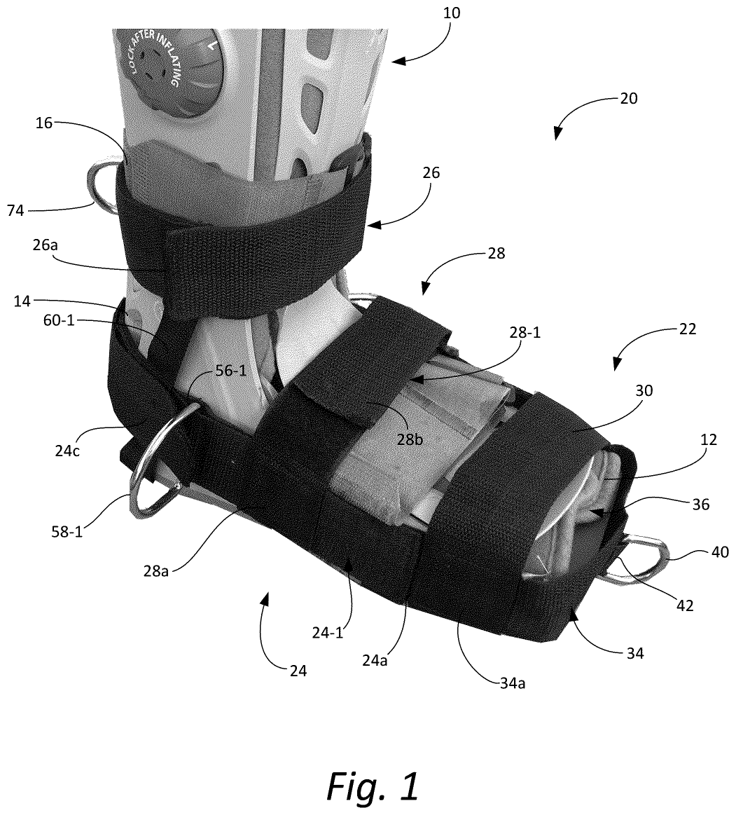

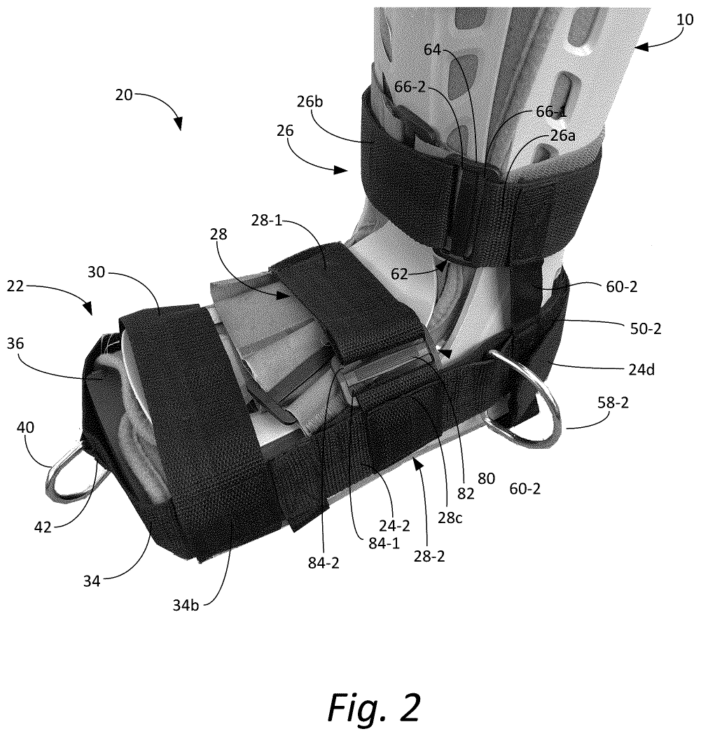

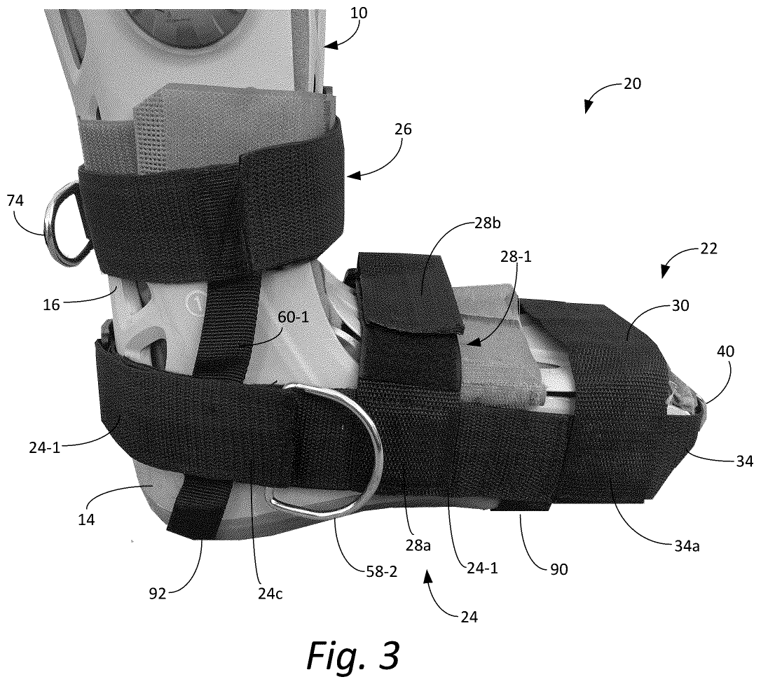

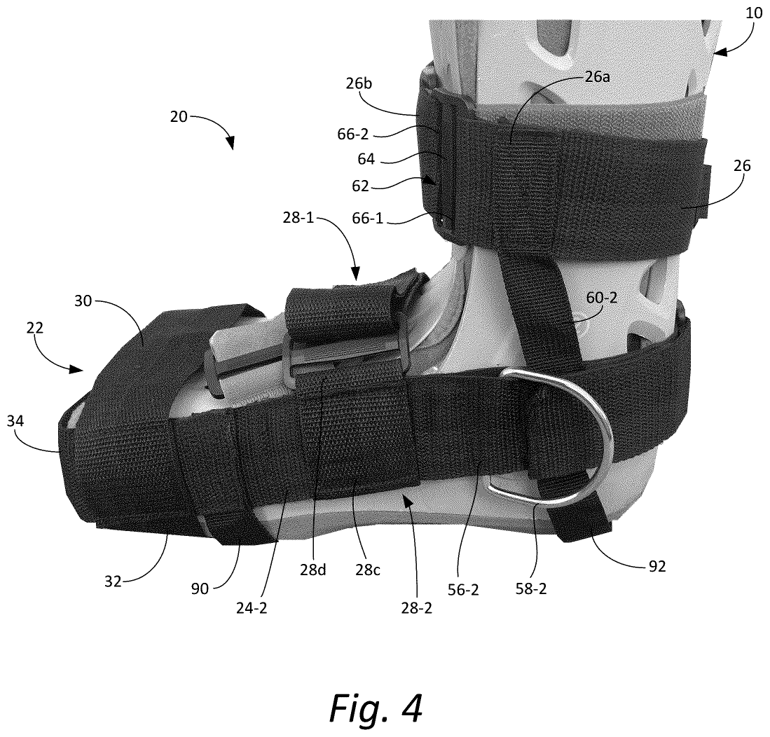

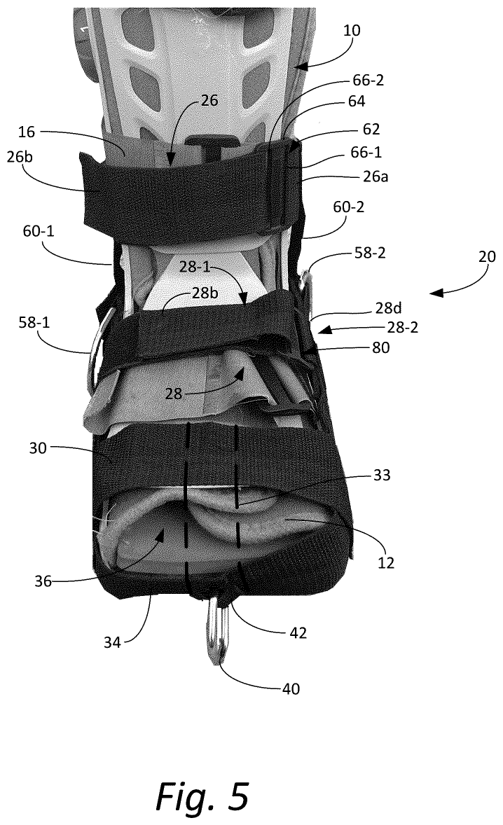

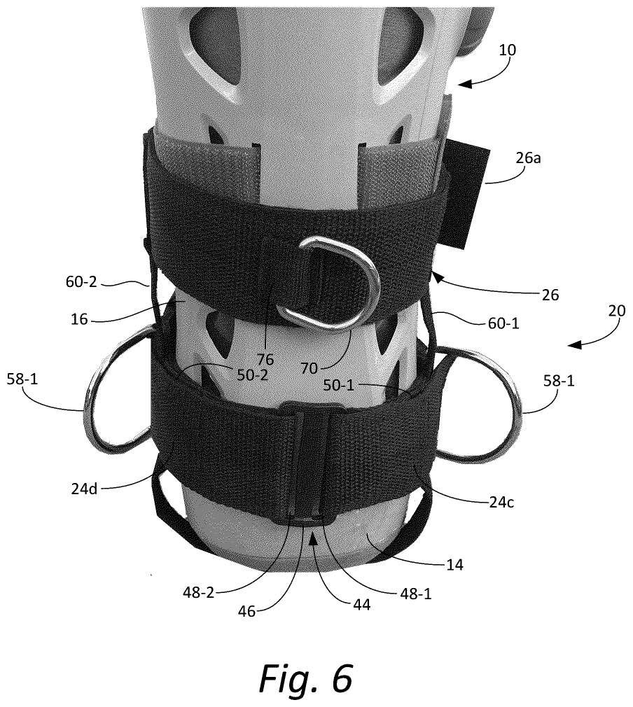

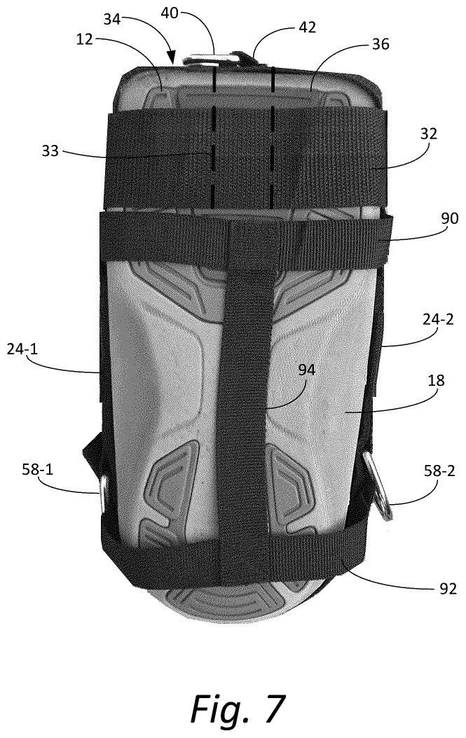

FIGS. 1-7 illustrate generally illustrate various views of an exercise rehabilitation harness 20, according to one example, which is configured to secure about different types of conventional orthotic boots, such as orthotic boot 10, to enable attachment to strength training equipment (e.g., to cables of resistance equipment) to aid in rehabilitation of lower leg injuries when a patient is wearing the orthotic boot. FIGS. 1 and 2 respectively illustrate front right and front left perspective views of, FIGS. 3 and 4 respectively illustrate right and left side views, FIGS. 5 and 6 respectively illustrate front and back side views, and FIG. 7 illustrates a bottom view of exercise rehabilitation harness 20. It is noted that the while the below description references each of the FIGS. 1-7, each of the identified elements of exercise rehabilitation harness 20 may appear in one or more, but not in each, of the FIGS. 1-7. For ease of description, it is further noted that exercise rehabilitation harness 20 is hereinafter referred to simply as harness 20. Similarly, orthotic boot 10 is referred to simply as boot 10.

With reference to FIGS. 1-7, according to examples, harness 20 includes a toe cap 22, a heel strap 24 including a first heel strap segment 24-1 and a second heel strap segment 24-2, an ankle strap 26, and a mid-strap 28. It is noted that, in some examples, harness 20 is configured to be universal, so as to be fitted over the boot in the same fashion whether boot 10 is on either the right foot or left foot of a user. For ease of description, it is noted that harness 20 is described herein in terms of harness 20 being secured to boot 10 when being employed on a user's right leg. In FIGS. 1-7, it is noted that if boot 10 is employed on a user's right leg, first heel strap segment 24-1 would extend along the lateral side and second heel strap segment 24-2 would extend along the medial side of a user's foot (and vice-versa when orthotic boot is employed on a user's left leg). In examples, the various straps described herein may be constructed of nylon, or other suitable materials (e.g., high tensile strength and flexible), such as neoprene, plastics, or leather, for example.

In one example, toe cap 22 includes an upper toe strap segment 30 and a lower toe strap segment 32 (see FIG. 7) which are respectively configured to extend over an upper and a lower side of a toe portion 12 of boot 10, and a forward toe strap segment 34 which is configured to extend laterally across toe portion 12 of boot 10. Together, upper toe strap segment 30, lower toe strap segment 32, and forward toe strap segment 34 form a toe cup 36 which is to receive and fit over toe portion 12 of orthotic boot 10. Together, upper and lower toe strap segments 30 and 32 are configured to extend about a circumference of toe portion 12 of boot 10. In one example, upper and lower toe strap segments 30 and 32 are part of a continuous segment which is configured to extend about the circumference of toe portion 12, wherein opposing sides of the continuous segment are attached to forward toe strap segment 34.

In examples, with reference to FIGS. 5 and 7, toe cap 22 may further include a vertical toe strap segment 33 (illustrated in heavy dashed lines) that extends vertically across toe portion 12 of orthotic boot 10 from upper toe strap segment 30 to lower toe strap segment 32. In examples, opposing ends of vertical toe strap segment 33 are respectively sewn to upper and lower toe strap segments 30 and 32, and a mid-region of vertical toe strap segment 33 is sewn to forward toe strap segment 34. Vertical toe strap segment 33 is configured to prevent vertical movement of connector element 40 relative to toe portion 12 of orthotic boot 10, and to assist in preventing toe portion 12 of orthotic boot 10 from slipping out of toe cap 22.

In one example, opposing ends 34a and 34b of forward toe strap segment 34 are disposed on opposing sides of the toe portion 12 of orthotic boot 12 (on lateral and medial sides of boot 12) and are attached to upper and lower toe strap segments 30 and 32, such as via sewing, for instance. According to one example, as illustrated, opposing ends 34a and 34b of forward toe strap segment 34 are positioned so as to be inside upper and lower toe strap segments 30 and 32 (so as to be positioned between upper and lower toe strap segments 30 and 32 and boot 10 when harness 20 is secured thereto). In other examples, opposing ends 34a and 34b of forward toe strap segment 34 may be positioned so as to be outside of upper and lower toe strap segments 30 and 32.

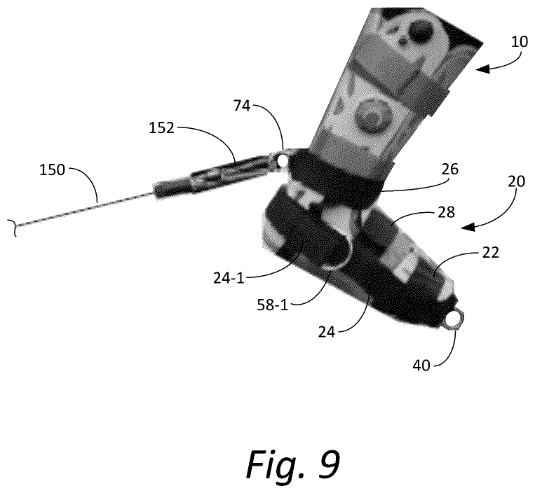

In one example, a connector element 40 is attached to forward toe strap segment 34, where connector element 40 is configured to connect to external exercise equipment, such as resistance training equipment (e.g., see FIG. 9). In one example, as illustrated, connector element 40 comprises a D-ring which is connected to forward toe strap segment 32 via a loop segment 42 sewn on forward toe strap segment 32, wherein a straight portion of the D-ring is passes through and is retained by loop segment 42 and is able to pivot therein. In addition to employing a D-ring, in other examples, any number of other suitable connector elements 40 may be employed, such as an O-ring, a carabineer, a hook, a fabric loop (e.g., loop segment 42), or any other device suitable for connecting to exercise equipment.

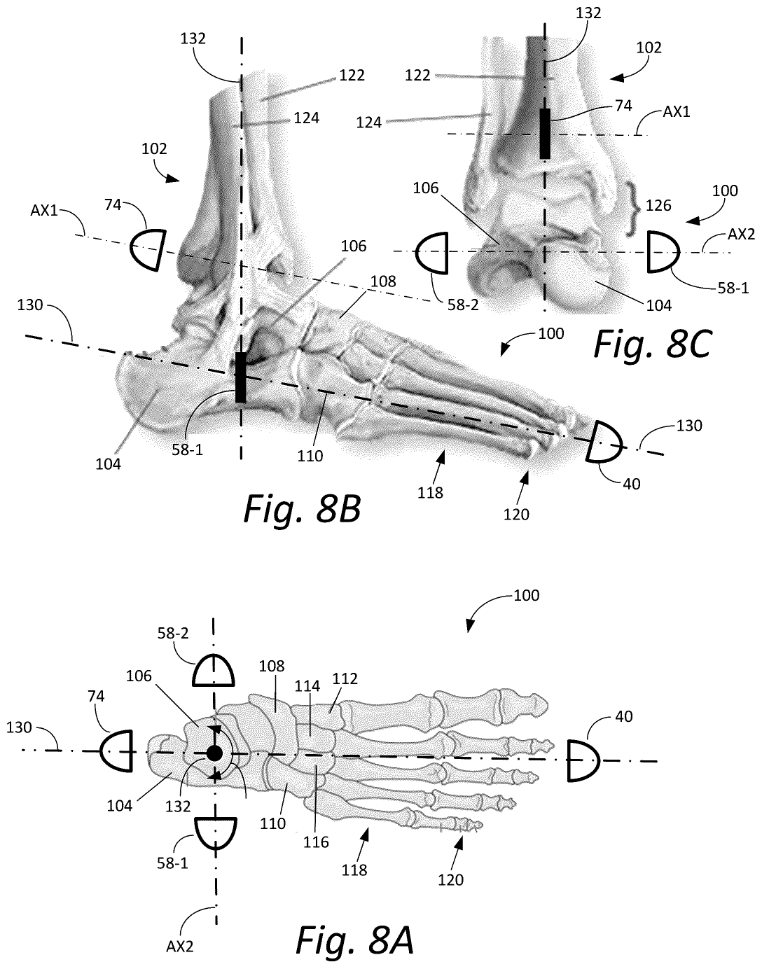

In accordance with the present disclosure, coupling point 40 is positioned to be forward of toe portion 12 of boot 10. More particularly, with reference to FIGS. 8A-8C, as will be described in greater detail below, when harness 20 is secured to boot 10 with a user's foot secured therein, connector element 40 is positioned so as to be disposed along a longitudinal axis 130 of a user's foot and forward of the toes (phalanges). By locating connector element 40 at such position, and by enabling connector element 40 to pivot relative to harness 20 (i.e., is pivotally connected thereto via loop segment 42), the potential of torsional forces being applied to boot 10 is greatly reduced, thereby greatly reducing the potential for unwanted twisting of portions of a user's lower leg (such as to the knee and ankle, for example) when using harness 20 for resistance training exercises for injury rehabilitation.

In one example, first and second heel strap segments 24-1 and 24-2 respectively have first ends 24a and 24b connected to opposing ends 34a and 34b of forward toe strap segment 34 of toe cap 22. In examples, first and second heel strap segments 24-1 and 24-2 respectively extend longitudinally in parallel along opposing sides a foot portion of boot 10 from first ends 24a and 24b at toe cap 22 to second ends 24c and 24d, where second ends 24c and 24d are configured to meet at and be interconnected by a coupling member 44. In one example coupling member 44 is configured to be disposed directly behind the heel of boot 10 when harness 20 is installed thereon.

With particular reference to FIG. 6, in one example, as illustrated, coupling member 44 comprises a double buckle arrangement where coupling member includes a frame element 46 defining first and second openings 48-1 and 48-2. When securing harness 20 to boot 10, free second ends 24c and 24d of first and second heel strap segments 24-1 and 24-2 respectively pass through first and second openings 48-1 and 48-2 of buckle frame 46. The free ends 24c and 24d of first and second heel strap segments 24-1 and 24-2 are then pulled outward and forward toward toe portion 12 of boot 10 which draws buckle frame 46 against the back of boot 10 and shortens a length of first and second heel strap segments 24-1 and 24-2 between coupling member 44 and toe cap 22. The shortening of first and second heel strap segments 24-1 and 24-2 acts to pull toe cap 22 toward coupling member 44 so that toe cap 22 is drawn onto boot 10 with toe cup 36 secured tightly about toe portion 12. After being tightened to secure toe cup 36 about toe portion 12, first and second heel strap segments 24-2 and 24-2 are secured via a securing mechanism 50 to remain in their tightened positions.

Employing a double buckle arrangement for coupling member 44 enables first and second heel strap segments 24-1 and 24-2 to be independently tightened (i.e., enables independent adjustment of the lengths of first and second heel strap segments 24-1 and 24-2 between coupling member 44 and toe cap 22). Such independent tightening of first and second heel strap segments 24-1 and 24-2 enables toe cap 22 to be evenly drawn over and secured to toe portion 12 of boot 12 so that connector element 40 of toe cap 22 remains centered on toe portion 12 (i.e., positioned on a longitudinal axis 130 of a user's foot forward of the phalanges).

In examples, after being tightened to secure toe cup 36 about toe portion 12, first and second heel strap segments 24-2 and 24-2 are secured via corresponding securing mechanisms 50-1 and 50-2 so as to remain in their tightened positions. In one example, such securing mechanisms 50-1 and 50-2 comprise a hook and loop type fastener (e.g., Velcro™), wherein a hook portion is disposed proximate to respective second ends 24c and 24d of first and second heel strap segments 24-1 and 24-2, and a loop portion is disposed on first and second heel strap segments 24-1 and 24-2 at a position between toe cap 22 and respective second ends 24c and 24d which aligns with the hook type portion when first and second heel strap segments 24-1 and 24-2 are in a tightened position. Additionally, suitable securing means other than a hook and loop type fastener may be employed, such as one or more snaps, clasps, and pinch connector, for example.

In addition to a double buckle type arrangement herein, coupling member 44 may comprise any number of other suitable coupling means which may, or may not, enable independent adjustment of first and second heel strap segments 24-1 and 24-2, including other types of buckles such as tongue buckles, clamp closure buckles, slide buckles, cam buckles, and roller buckles, for example.

In examples, first and second heel strap segments 24-1 and 24-2 respectively define loop segments 56-1 and 56-2 which respectively retain connector elements 58-1 and 58-2 for connecting to exercise equipment. In one example, as illustrated, similar to that described above with respect to connector element 40, connector elements 58-1 and 58-2 comprise D-rings having a flat side retained within corresponding loop segments 56-1 and 56-2. In examples, loop segments 56-1 and 56-2 and corresponding D-rings 58-1 and 58-2 are positioned so as to be aligned in the horizontal direction with a longitudinal axis 132 of lower leg 102 (extending along the tibia and fibula bones) when harness 20 is secured to boot 10 (see FIGS. 8A-8C). By locating connector elements 58-1 and 58-2 at such positions, and by enabling connector elements 58-1 and 58-2 to pivot relative to harness 20 (i.e., is pivotally connected thereto via loop segments 56-1 and 56-2), the potential of torsional forces being applied to a user's lower leg is reduced, thereby reducing the potential for unwanted twisting of portions of a user's lower leg (such as to the knee and ankle, for example) when using connector element 58-1 and 58-2 of harness 20 for connection to exercise equipment to perform resistance training exercises for injury rehabilitation.

In examples, ankle strap 26 extends between a first end 26a and a second end 26b and is configured to wrap around and secure to an ankle portion 16 of boot 10 when harness 20 is secured thereto. In one examples, ankle strap 26 is respectively connected to first and second heel strap segments 24-1 and 24-2 via connector straps 60-1 and 60-2, where a length of connector straps 60-1 and 60-2 is configured to vertically position ankle strap 26 at a predetermined selected location about ankle portion 16 of boot 10 (e.g., above an ankle joint of a user's foot when inserted in boot 10). In other examples, ankle strap 26 may be connected to heel strap 24 using more or fewer than two connector straps (e.g., at least one connector strap 60).

In one example, a buckle 62 having a frame 64 defining first and second frame openings 66-1 and 66-2 is employed to tighten ankle strap 26. According to one example, first end 26a of ankle strap 26 is connected to buckle 62 via first frame opening 66-1. To secure ankle strap 26 to boot 10, the second end 26b is passed through second frame opening 66-2 of buckle 62 and pulled back on itself which draws buckle frame 64 against boot 10 and shortens a length of ankle strap 26 extending about ankle portion 16 of boot 10 between first and second frame opening 66-1 and 66-2. After being securely tightened about ankle portion 16 of boot 10, ankle strap 26 is secured via a securing mechanism 70 to remain in its tightened positions. In examples, securing mechanism 70 is similar to securing mechanisms 50-1 and 50-2 described above for securing first and second heel strap segments 24-1 and 24-2. In one example, securing mechanism 70 comprises a hook and loop type fastener (e.g., Velcro™), wherein a hook portion is disposed proximate to respective second end 26b of ankle strap 26, and a loop portion is disposed on ankle strap 26 at a position between first and second frame openings 66-1 and 66-2 which aligns with the hook type portion when ankle strap 26 is in the tightened position. In other examples, ankle strap 26 may employ a double buckle arrangement similar to that employed with respect to first and second heel strap segments 24-1 and 24-2 so that each end of ankle strap 26 may be adjusted to thereby provide positioning of connector element 74 on the backside of boot 10.

In one example, a connector element 74 is connected to ankle strap 26 via a loop segment 76 sewn thereon. In one example, as illustrated, connector element 74 is D-ring having a straight portion which passes through and is retained by loop segment 76 such that D-ring 74 is able to pivot therein. According to one example, in accordance with the present disclosure, connector element 74 and corresponding loop segment 76 are positioned so as to be located directly on the back of boot 10 and to be aligned in the horizontal direction with a longitudinal axis 132 of lower leg 102 (extending along the tibia and fibula bones) and be positioned above the ankle joint of a user's foot when harness 20 is secured to boot 10 (see FIGS. 8A-8C). By locating connector element 74 at such position, and by enabling connector element 74 to pivot relative to harness 20 (i.e., is pivotally connected thereto via loop segment 76), the potential of torsional forces being applied to a user's lower leg is reduced, thereby reducing the potential for unwanted twisting of portions of a user's lower leg (particularly to the knee and ankle) when employing connector element 74 of harness 20 for performing resistance training exercises for injury rehabilitation. Furthermore, by employing separate heel and ankle straps 24 and 26, the positions of connector element 40 of toe cap 22 and connector element 74 of ankle strap 26 can independently adjusted so that the positioning of connector element 40 does not impact the positioning of connector element 74 (and vice-versa).

With specific reference to FIG. 7, harness 20 further includes a plurality of bottom straps including a first bottom strap 90, a second bottom strap 92, and a third bottom strap 94, where the plurality of bottom straps are arranged to engage a bottom side 18 of boot 10 when harness 20 is secured thereto. In one example, first bottom strap 90 has opposing ends 90a and 90b which are respectively connected to first and second heel strap segments 24-1 and 24-2, with first bottom strap 90 configured to extend laterally across the bottom 18 of boot 12 adjacent to lower toe strap segment 32 of toe cap 22. In one example, second bottom strap 92 has opposing ends 92a and 92b which are respectively connected to first and second heel strap segments 24-1 and 24-2, with second bottom strap 92 configured to extend laterally across the bottom 18 of boot 10 below heel portion 14. In one example, third bottom strap 94 is connected between first and second bottom straps 90 and 92 and extends longitudinally there between across the bottom 18 of boot 10. In other examples (not illustrated), first bottom strap 90 may be eliminated with third bottom strip being connected between second bottom strap 92 and lower toe strap segment 32.

In examples, mid-strap 28 includes a first mid-strap segment 28-1 and a second mid-strap segment 28-2. In one example, as illustrated, first mid-strap segment 28-1 has a first end 28a connected to first heel strap segment 24-1 (e.g., via sewing) and a free second end 28b. Second mid-strap segment 28-2 has a first end 28c connected to second heel strap segment 24-2 and a second end 28d connected to a buckle 80. In one example, as illustrated, buckle 80 includes a buckle frame 82 defining first and second frame openings 84-1 and 84-2, with second end 28d of second mid-strap segment 28-2 is connected to buckle 80 via second frame opening 84-2, and first frame opening 84-1 to receive second end 28b of first mid-strap segment 28-1.

Similar to that described above with respect to ankle strap 26, to tighten mid-strap 28, second end 28b of first mid-strap segment 28-1 is passed through first frame opening 84-1 of buckle 80 and pulled back onto itself, which draws buckle frame 82 against boot 10 and shortens a length of first mid-strap segment 28-1 extending between first heel strap segment 24-1 and buckle 80. With harness 20 installed on boot 10, tightening of mid-strap 28 pulls first and second heel strap segments 24-1 and 24-2 upward which, in-turn, pulls first and second bottom straps 90 and 92 upward and tightly against bottom 18 of boot 10, and also pulls toe cap 22 and buckle 46 of heel strap 24 toward mid-strap 28 which thereby draws toe cap 22 more tightly onto toe portion 12 of boot 10 and heels strap 28 more tightly against the back of heel 14 of boot 10.

After being securely tightened about boot 10, mid-strap 28 is secured via a securing mechanism 88 to remain in its tightened positions. In examples, securing mechanism 88 is similar to securing mechanisms 50-1 and 50-2 described above for securing first and second heel strap segments 24-1 and 24-2. In one example, securing mechanism 88 comprises a hook and loop type fastener (e.g., Velcro™), wherein a hook portion is disposed proximate to respective second end 28b of first mid-strap segment 28-1, and a loop portion is disposed on first mid-strap segment 28-1 at a position between first end 28b and buckle 80 which aligns with the hook type portion when ankle strap 26 is in the tightened position.

FIG. 8A illustrates a top view of the skeletal structure of a human foot 100. FIGS. 8B and 8C respectively illustrate lateral and anterior views of the skeletal structure of human foot 100 and portions of the lower leg 102. FIGS. 8A-8C describe the positioning of connector elements 40, 58-1, 58-2, and 74 (e.g., D-rings) of harness 20 relative to the skeletal structure of the lower leg and foot when hardness 20 is secured to boot 10.

Human foot 100 includes the calcaneus bone 104, the talus bone 106, the navicular bone 108, the cuboid bone 110, the medial, intermediate, and lateral cuneiform bones 112, 114, 116, the metatarsal bones 118, and phalanges bones 120. Lower leg 102 includes a tibia bone 122 and fibula bone 124, with the ankle joint being illustrated at 126. In one example, a longitudinal axis 130 can be said to extend through foot 100 from phalanges bones 120 to calcaneus bone 104, and a vertical axis 132 can be said to extend through lower leg 102 along the tibia and fibula bones 122 and 124.

As described above, when harness 20 is secured to boot 10, connector element 40 is arranged to be located along longitudinal axis 130 of the foot 100, directly forward of and centered on phalanges 120. Connector element 74 is arranged to be located vertically in-line with longitudinal axis 130 and to be disposed vertically above ankle joint 126, as illustrated by axis, AX1, where axis AX1 is in parallel with longitudinal axis 130. Connector elements 58-1 and 58-2 are disposed on opposite sides of foot 100 and arranged to be located along an axis AX2 which is horizontally in-line with vertical axis 132 of lower leg 102, and which is perpendicular to both longitudinal axis 130 of foot 100 and vertical axis of lower leg 102. In one example, axis AX2 is disposed vertically below ankle joint 126.

As described above, positioning connector elements 40, 58-1, 58-2, and 74 relative to longitudinal axis 130 of foot 100 and vertical axis 132 of lower leg 102, as described herein, reduces the potential for generating torsional forces 140 and 142 about longitudinal axis 130 of foot 100 and vertical axis 132 of lower leg 102 when harness 20 is secured to boot 10 and being employed by a user to perform resistance training exercises using exercise equipment connected to harness 20 via connector elements 40, 58-1, 58-2, and 74 (e.g., see FIG. 9). Reducing the potential of such torsional forces 140 and 142, in-turn, reduces the possibility of potential twisting of the lower leg and foot during resistance training exercises and, thus, reduces the possibility of aggravating existing injuries, or causing additional injury, during the injury rehabilitation process. As such, rehabilitation harness 20, in accordance with the present disclosure, enables a user to perform strength training exercises when wearing an orthotic boot to avoid muscle atrophy while also reducing and/or eliminating the potentially for aggravating existing injuries or causing additional injury. By enabling a user to prevent muscle atrophy, rehabilitation harness 20 enables quicker and improved recovery from lower leg injuries. While known foot harnesses are available, such known harnesses are explicitly designed to fit directly onto a foot or over an athletic shoe, and are not designed for, or even capable of, use with orthotic boots nor intended for injury rehabilitation as placement of strap and connector elements for connecting to exercise equipment is not made with the intent to eliminate torsional forces on the lower leg and foot.

FIG. 9 is a side view generally illustrating exercise rehabilitation harness 20 secured to an orthotic boot 10, according to one example. FIG. 9 further illustrates a cable 150, such as from a weight stack machine (not illustrated), connected to rear connector element 74, such as via a carabiner type connector 152, for example. Such connections can be made in a similar fashion to other connector elements (e.g., connector elements 40, 58-1, and 58-1) of exercise rehabilitation harness 20. Although illustrated as being connected to a weight stack machine via a cable connection, it is noted that the connector elements may be connected to any number of different types of rehabilitation equipment including elastic athletic bands, and Keiser™ machines, for example.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. An exercise rehabilitation harness to secure about an orthotic boot, the harness comprising:
a toe cap defining a toe cup to receive a toe portion of the orthotic boot;
a heel strap including:
a heel strap coupling member to be disposed on a backside of a heel portion of the orthotic boot;
a first heel strap segment and a second heel strap segment, the first and second segments each having a first end connected to the toe cap, the first and second heel strap segments extending longitudinally in parallel with one another on opposite sides of a foot portion of the orthotic boot to second ends, the heel strap coupling member to interconnect the second ends on the backside of the heel portion of the orthotic boot; and
an ankle strap including:
an ankle strap coupling member;
an ankle strap segment having opposing first and second ends, the ankle strap segment to wrap around and secure to an ankle portion of the orthotic boot vertically above the heel of the orthotic boot, the ankle strap coupling member to interconnect the opposing first and second ends; and
an backside connector element attached to the ankle strap segment so as to be disposed on a backside of an ankle portion of the orthotic boot, the backside connector element to connect to exercise equipment; and
at least one connector strap which connects the ankle strap segment to one of the first and second heel strap segments.

2. The exercise rehabilitation harness of claim 1, when the exercise harness is secured to the orthotic boot and a lower leg and foot of a user are secured within the boot, the backside connector element to be positioned to be positioned vertically above an ankle joint of the foot and in-line with a longitudinal access of the foot and with a longitudinal axis of the lower leg.

3. The exercise rehabilitation harness of claim 2, the at least one connector strap having a length to position the ankle strap segment and backside connector element at a predetermined vertical position on the ankle portion of the orthotic boot.

4. The exercise rehabilitation harness of claim 1, the backside connector element comprising a ring element.

5. The exercise rehabilitation harness of claim 1, wherein the toe cap includes a toe connector element to be positioned at a location horizontally forward of the toe portion of the orthotic boot, the toe connector element to connect to exercise equipment.

6. The exercise rehabilitation harness of claim 6, when the exercise harness is secured to the orthotic boot and a lower leg and foot of a user are secured within the boot, the toe connector element to be positioned along a longitudinal access of the foot and be centered and forward of phalanges of the foot.

7. The exercise rehabilitation harness of claim 1, the heel strap coupling member to enable independent tightening of the first and second heel strap segments.

8. The exercise rehabilitation harness of claim 7, the heel strap coupling member comprising a double buckle arrangement to enable independent tightening of the first and second heel strap segments.

9. The exercise rehabilitation harness of claim 1, further including:
a mid-strap disposed between the toe cap and the heel strap coupling member, the mid-strap to extend over an upper side of the orthotic boot forward of an ankle portion of the orthotic boot, the mid-strap including:
a mid-strap coupling member;
a first mid-strap having a first end attached to first heel strap segment and a free second end; and
a second mid-strap segment having a first end attached to the second heel strap segment and a second end attached to the mid-strap coupling member, the mid-strap coupling member to receive the second end of the first mid-strap segment to interconnect the first and second mid-strap segments.

10. The exercise rehabilitation harness of claim 1, including:
a first bottom strap attached between the first and second heel strap segments, the first bottom strap to extend laterally across a bottom of the orthotic boot proximate to the toe portion of the boot;
a second bottom strap attached between the first and second heel strap segments, the second bottom strap to extend laterally across a bottom heel portion of the orthotic boot; and
a third bottom segment attached between the first and second bottom straps, the third bottom segment to extend longitudinally across the bottom of the orthotic boot.

11. An exercise rehabilitation harness to secure about an orthotic boot, the harness comprising:
a toe cap defining a toe cup to receive and fit over a toe portion of the orthotic boot, the toe cap including a toe connector element disposed at a location such that when the exercise harness is secured to the orthotic boot and a lower leg and foot of a user are secured within the boot, the toe connector element is to be positioned along a longitudinal access of the foot and be centered and forward of phalanges of the foot, the toe connector element to connect to exercise equipment; and
a heel strap including
a heel strap coupling member to be disposed on a backside of a heel portion of the orthotic boot;
a first heel strap segment and a second heel strap segment, the first and second segments each having a first end connected to the toe cap, the first and second heel strap segments to extend longitudinally in parallel with one another on opposite sides of a foot portion of the orthotic boot to second ends, the heel strap coupling member to interconnect the second ends on the backside of the heel portion of the orthotic boot.

12. The exercise rehabilitation harness of claim 11, the toe cap including:
an upper toe strap segment to extend over an upper side of a toe portion of the orthotic boot;
a lower toe strap segment to extend across a lower side of the toe portion of the boot; and
a forward toe strap segment to across a front side of the toe portion of the boot, wherein the upper, lower, and forward toe strap segments together form the cup, and wherein the toe connector element is attached to the forward toe strap segment.

13. The exercise rehabilitation harness of claim 11, the heel strap coupling member to enable independent adjustment of a length of the first and second heel strap segments to enable independent tightening of the first and second heel strap segments.

14. The exercise rehabilitation harness of claim 11, further comprising:
an ankle strap including:
an ankle strap coupling member;
an ankle strap segment having opposing first and second ends, the ankle strap segment to wrap around and secure to an ankle portion of the orthotic boot vertically above the heel of the orthotic boot, the ankle strap coupling member to interconnect the opposing first and second ends; and
an backside connector element attached to the ankle strap segment so as to be disposed on a backside of an ankle portion of the orthotic boot, the backside connector element to connect to exercise equipment; and
at least one connector strap which connects the ankle strap segment to one of the first and second heel strap segments, the at least one connector strap having a length to position the ankle strap segment and backside connector element at a predetermined vertical position on the ankle portion of the orthotic boot.

15. The exercise rehabilitation harness of claim 14, when the exercise harness is secured to the orthotic boot and a lower leg and foot of a user are secured within the boot, the backside connector element to be positioned to be positioned vertically above an ankle joint of the foot and in-line with a longitudinal access of the foot and with a longitudinal axis of the lower leg.

16. The exercise rehabilitation harness of claim 11, further including:
a mid-strap disposed between the toe cap and the heel strap coupling member, the mid-strap to extend over an upper side of the orthotic boot forward of an ankle portion of the orthotic boot, the mid-strap including:
a mid-strap coupling member;
a first mid-strap having a first end attached to first heel strap segment and a free second end; and
a second mid-strap segment having a first end attached to the second heel strap segment and a second end attached to the mid-strap coupling member, the mid-strap coupling member to receive the second end of the first mid-strap segment to interconnect the first and second mid-strap segments.

17. The exercise rehabilitation harness of claim 11, further including:
a first bottom strap attached between the first and second heel strap segments, the first bottom strap to extend laterally across a bottom of the orthotic boot proximate to the toe portion of the boot;
a second bottom strap attached between the first and second heel strap segments, the second bottom strap to extend laterally across a bottom heel portion of the orthotic boot; and
a third bottom segment attached between the first and second bottom straps, the third bottom segment to extend longitudinally across the bottom of the orthotic boot.

18. The exercise rehabilitation harness of claim 11, wherein at least the heal strap coupling member is selected from the group consisting of a buckle, a snap lock, a clip, and a hook and loop fastener.
